(19) United States
(12) Patent Application Publication
(10) Pub. No.: US 2012/0228137 A1
(43) Pub. Date: Sep. 13, 2012

(54) pH SENSOR

(76) Inventors: Sung Kwon Cho, Pittsburgh, PA (US); William Walker Clark, Wexford, PA (US)

(21) Appl. No.: 13/510,450

(22) PCT Filed: Aug. 18, 2010
(86) PCT No.: PCT/US2010/045847

§ 371 Date: May 17, 2012

Related U.S. Application Data
(60) Provisional application No. 61/262,815, filed on Nov. 19, 2009.

(51) Int. Cl. G01N 27/30; A61B 5/145; F17D 1/08

(52) U.S. Cl. 204/409; 204/433; 137/561 A; 137/13; 600/345

(57) ABSTRACT

A pH sensor includes an enclosed fluidic channel, an electrolyte solution within the fluidic channel, a first electrode exterior to the fluidic channel, a second electrode within the fluidic channel, a liquid junction extending between the fluidic channel and an exterior of the fluidic channel. The liquid junction is adapted to provide fluid connection between the electrolyte solution within the fluidic channel and an exterior of the fluidic channel. The pH sensor further includes a fluidic switch or fluidic controller in operative connection with the liquid junction to control whether the liquid junction provides fluid connection between the electrolyte solution within the fluidic channel and the exterior of the fluidic channel.

### CROSS-REFERENCE TO RELATED APPLICATIONS

This application claims benefit of U.S. Provisional Patent Application No. 61/262,815 filed Nov. 19, 2009, the disclosure of which is incorporated herein by reference.

### BACKGROUND

The following information is provided to assist the reader to understand the technology described below and certain environments in which such technology can be used. The terms used herein are not intended to be limited to any particular narrow interpretation unless clearly stated otherwise in this document. References set forth herein may facilitate understanding of the technology or the background thereof. The disclosure of all references cited herein are incorporated by reference.

A typical pH sensor based on potentiometric principles includes a reference electrolyte solution, an indicating electrode immersed in or in contact with an analyte solution (of which the pH is to be measured), a reference electrode immersed in the reference electrolyte solution, and measurement circuitry such as potentiometric circuitry in electrical connection with the reference electrode and the indicating electrode. The potentiometric circuitry measures the electrical difference between the indicating and reference electrodes. Ionic contact between the electrolyte solutions in which the indicating electrode and the reference electrodes are immersed provides electrical connection between the electrodes. The pH value of the sample or analyte electrolyte solution (which is proportional to concentration of the hydrogen ions in the sample electrolyte) is directly correlated with the potential difference developed at the indicating electrode following the Nernst equation.

In the above-described configuration, an important condition for correct measurement is that the electric potential difference built up in the reference electrode and the reference electrolyte is maintained constant such that the reading from the potentiometric circuitry solely represents the potential difference in the indicating electrode, that is, pH in the electrolyte solution. To meet this condition, a common arrangement is to have the reference electrode immersed in a saturated reference electrolyte solution, and to have a small “window” positioned between the saturated reference electrolyte solution and the sample or analyte electrolyte solution to provide ionic contact and thus an electrical connection between the saturated reference electrolyte solution and the sample or analyte electrolyte solution. The “window” is usually fabricated from a porous material such as a porous glass membrane, a hydrophilic porous polymer membrane, etc. Because of the porosity of the “window”, a non-negligible mass exchange occurs between the saturated reference electrolyte solution and the sample or analyte electrolyte solution, thereby causing cross-contamination in both solutions.

The dilution of the saturated reference electrolyte solution resulting from such contamination can be a significant problem since it changes the potential difference in the reference electrode. The contamination also deteriorates the stability of the pH sensor and shortens the lifetime of the pH sensor. As the dimensions of a pH sensor are reduced (for example, to very small, microlevel, microscale or smaller dimension), the problem is exacerbated because the volume of the saturated reference electrolyte solution is very small compared to the sample electrolyte solution. For example, for applications where a microscale or smaller pH sensor is implanted into a human body and is utilized to measure a physiological pH (for example, myocardial pH), the volume of the saturated reference electrolyte solution is extremely small compared to the volume of the myocardial tissue of which the pH is to be measured. At such a scale, the saturated reference electrolyte solution is diluted much more quickly than in a macro scale glass tube type pH sensor.

Another factor which affects the useful life of a pH sensor such as a microscale pH sensor is the durability of the reference electrode. In many instances, conductive material of the reference electrode is gradually dissolved and consumed into the saturated reference electrolyte solution. At some point during the dissolution and consumption of the reference electrode, the useful life of the pH sensor is terminated.

### SUMMARY

In one aspect, a pH sensor includes an enclosed fluidic channel, an electrolyte solution within the fluidic channel, a first electrode exterior to the fluidic channel, a second electrode within the fluidic channel, and a liquid junction extending between the fluidic channel and an exterior of the fluidic channel. The liquid junction is adapted to provide fluid connection between the electrolyte solution within the fluidic channel and an exterior of the fluidic channel. The pH sensor further includes a fluidic switch or fluidic controller in operative connection with the liquid junction to control whether (or to the extent to which) the liquid junction provides fluid connection between the electrolyte solution within the fluidic channel and the exterior of the fluidic channel. The pH sensor can, for example, include a substrate and a cover connected to the substrate, wherein the cover and the substrate cooperate to define the fluidic channel.

The fluidic switch can, for example, include at least a first bubble within the fluidic channel. The first bubble can, for example, include a fluid immiscible in the reference electrolyte solution. In a number of embodiments, the pH sensor includes a bubble transportation system to transport the first bubble between a first position wherein it contacts the liquid junction and a second position wherein it does not contact the liquid junction. The first bubble can, for example, contact the liquid junction and the second electrode in the first position and not contact the liquid junction or the second electrode in the second position. In a number of embodiments, the first bubble forms a barrier between the liquid junction and electrolyte solution and forms a barrier between the second electrode and the electrolyte solution in the first position. In such embodiments, the bubble does not form a barrier between the liquid junction and electrolyte solution and does not form a barrier between the second electrode and the electrolyte solution in the second position. The bubble transportation system can, for example, include an electrowetting-on-dielectric system including an array of electrodes.

In a number of embodiments, the pH sensor can, for example, include an electrode system in fluid connection with the reference electrolyte solution within the fluidic channel to generate the first bubble within the fluidic channel.

The fluidic switch can, for example, include at least a second bubble spaced from the first bubble and hydrodynamically connected to the first bubble via the reference solution.

In a number of embodiments, the pH sensor can, for example, include an electrode system in fluid connection with the reference electrolyte solution within the fluidic channel to generate at least one bubble within the fluidic channel so that the bubble can contact the liquid junction and a system to reduce the size of the bubble so that the bubble does not contact the liquid junction. The system to reduce the size of the bubble can, for example, include a catalyst on the electrode system. The bubble can, for example, be generated to contact the liquid junction and the second electrode in the first position and then reduced in size so that the bubble does not contact the liquid junction or the second electrode. In a number of embodiments, the electrode system generates at least one bubble within the fluidic channel so that the bubble can form a barrier between the liquid junction and electrolyte solution and form a barrier between the second electrode and the electrolyte solution. In such embodiments, the system to reduce the size of the bubble reduces the size of the bubble so that the bubble does not form a barrier between the liquid junction and electrolyte solution and does not form a barrier between the second electrode and the electrolyte solution.

The substrate of the pH sensor can, for example, include a glass or a polymer. The cover of the pH sensor can, for example, include a glass or a polymer. In a number of embodiments, the cover or the substrate includes polydimethylsiloxane. The first electrode can, for example, include at least one of platinum, chromium, titanium, or iridium oxide. The second electrode can, for example, include at least one of platinum, chromium, titanium, silver, and silver chloride.

The reference solution can, for example, include a compound such as a salt that dissociates into ions in solution. The reference electrolyte can, for example, include at least one of a potassium chloride solution or a silver chloride solution. The liquid junction can, for example, include a porous polymer.

The pH sensor can, for example, be a microscale or smaller (for example, nanoscale) pH sensor. In a number of embodiments, the pH sensor has dimensions (that is, height, width and length) less than one centimeter. The pH sensor can, for example, be adapted to be implantable within a body.

In another aspect, a fluidic controller, includes an enclosed fluidic channel, a liquid within the fluidic channel, a liquid junction extending between an interior of the fluidic channel and an exterior of the fluidic channel and at least a first bubble within the fluidic channel. The extent to which the bubble contacts the liquid junction determine the extent to which the liquid within the fluidic channel is in fluid connection with the exterior of the fluidic channel. The bubble (which can be immiscible in the liquid) can, for example, encompass that portion of the liquid junction within the fluidic channel or form a barrier between the liquid junction and the liquid within the fluidic channel, thereby preventing fluid connection between the liquid and the exterior of the fluidic channel.

The fluidic controller claim 23 further comprising a bubble transportation system to transport the first bubble between a first position wherein it contacts the liquid junction and a second position wherein it does not contact the liquid junction. The bubble transportation system can, for example, include an electrowetting-on-dielectric system comprising an array of electrodes. The fluidic controller can further include an electrode system in fluid connection with the reference electrolyte solution within the fluidic channel to generate the first bubble within the fluidic channel.

In a number of embodiments, the fluidic controller includes an electrode system in fluid connection with the liquid within the fluidic channel to generate the first bubble within the fluidic channel so that the first bubble can contact the liquid junction and a system to reduce the size of the bubble so that the first bubble does not contact the liquid junction. The system to reduce the size of the bubble can, for example, include a catalyst on the electrode system.

In a further aspect, a method of controlling fluid connection between a liquid in an enclosed fluidic channel and an exterior of the fluidic channel, wherein the fluidic channel includes a liquid junction extending between the fluidic channel and the exterior of the fluidic channel and the liquid junction is adapted to provide fluid connection between fluidic channel and the exterior of the fluidic channel, includes: controllably positioning a bubble of a fluid immiscible in the liquid in contact with the liquid junction. The bubble can, for example, be positioned in contact with the liquid junction to remove the liquid from fluid connection with the exterior of the fluidic channel. The bubble can, for example, be removed (or partially removed) from contact with the liquid junction to place the liquid in fluid connection with the exterior of the fluidic channel. In other words, the bubble is controllably or selectably positionable to contact the liquid junction. The bubble can, for example, form a barrier between the liquid junction and the liquid when in contact with the liquid junction to substantially or completely remove the liquid from fluid connection with the exterior of the fluidic channel. In the case that the liquid is an ionically conductive liquid and the exterior of the fluidic channel includes an ionically conductive liquid, the method can provide an electrical on/off switch.

The technology described herein, along with the attributes and attendant advantages thereof, will best be appreciated and understood in view of the following detailed description taken in conjunction with the accompanying drawings in which representative embodiments are described by way of example.

### BRIEF DESCRIPTION OF THE DRAWINGS

FIG. 1 illustrates a top view of an embodiment of a pH sensor.

FIG. 2 illustrates a cross-sectional view of the pH sensor of FIG. 1 along A-A′ illustrated in FIG. 1.

FIG. 3 illustrates a cross-sectional view of another embodiment of a pH sensor.

FIG. 4 illustrates a cross-sectional view of another embodiment of a pH sensor.

### DETAILED DESCRIPTION

As used herein and in the appended claims, the singular forms “a,” “an”, and “the” include plural references unless the content clearly dictates otherwise. Thus, for example, reference to “a bubble” includes a plurality of such bubbles and equivalents thereof known to those skilled in the art, and so forth, and reference to “the bubble” is a reference to one or more such bubbles and equivalents thereof known to those skilled in the art, and so forth.

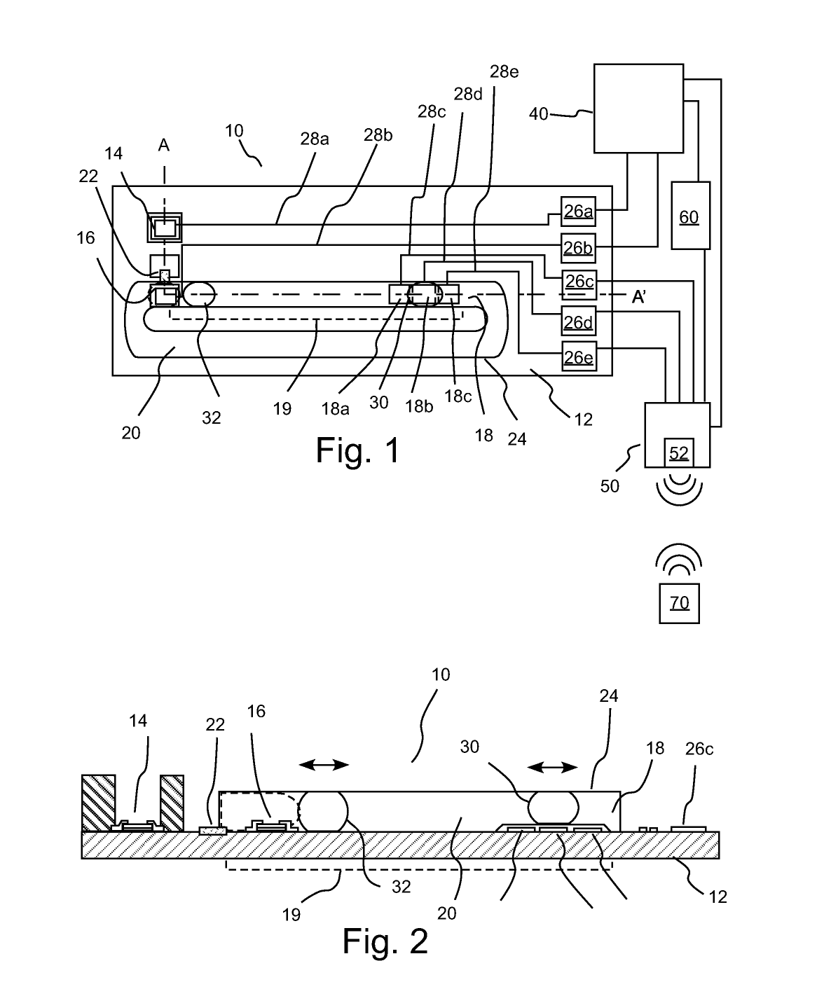

FIG. 1 illustrates a top view of a pH sensor 10 according to various embodiments which is readily formed to microscale or smaller (for example, nanonscale) dimensions. The term “microscale” as used in connection with the pH sensor hereof refers to sensors having dimensions smaller than one centimeter. In a number of embodiments, the dimensions of the pH sensors hereof are amenable to micro- and/or nanofabrication techniques. In a number of embodiments, the reference electrolyte solution volume was 20 cubic mm or less.

FIG. 2 illustrates a cross-sectional view of pH sensor 10 (taken along line A-A′ illustrated in FIG. 1). In a number of embodiments. pH sensor 10 can, for example, be formed in a size and configuration which allows for its implantation into a body (that is, within a human or animal) using a minimally invasive technique. In a number of embodiments, the length, width and height of pH sensor were each less than 1 centimeter. Such a microscale pH sensor 10 may, for example, be used in a variety of applications to measure the pH of a sample electrolyte, a sample tissue, etc. Such applications include medical applications where the microscale pH sensor 10 is utilized to measure the pH of myocardial tissue, brain tissue, liver tissue, kidney tissue, lung tissue, etc.

In the representative embodiment of FIGS. 1 and 2, pH sensor 10 includes a substrate 12, a first electrode 14, a second electrode 16, a system for transporting a bubble 18, a fluidic closed loop channel 20, a liquid junction 22, a cover 24, a plurality of connection pads 26a through 26e, and a plurality of conductors 28a through 28e (see FIG. 1).

Substrate 12 may, for example, include any suitable type of material that is, for example, amenable to fabrication of the various electrodes and other layers that it supports. Suitable materials include, for example, silicon-based materials (for example, silicon, glass etc.), non-silicon-based materials, polymeric materials (for example, polydimethylsiloxane or PDMS) and other materials. In the case the sensor is to be implantable within a body, the material can, for example, be bio-compatible. In a number of embodiments, for example, substrate 12 is a glass substrate. The first electrode 14 functions as an indicating or sensing electrode, and may, for example, include any suitable type of material. In general, it is desirable that the material for first electrode 14 exhibit a wide pH response range, high sensitivity, fast response time, low potential drift, in sensitivity to stirring, a wide temperature operating range and a wide operating pressure range.

First electrode 14 can, for example, include an ion-selective field effect transistor (ISFET) or a metal oxide electrode. An ISFET is part of a solid-state integrated circuit. The ISFET exhibits a fast response time (on the order of 1 millisecond) and is quite rugged in in-vivo applications.

In the case of a metal oxide electrode, a number of metal oxides are suitable for use in first electrode 14. Metal oxides can, for example, be deposited upon a conductive (for example, metallic) layer that is deposited or formed on substrate 12. A metal oxide film or layer (for example, iridium oxide) can, for example, be created via a variety of techniques including electrochemical oxidation via potential cycling, reactive sputtering, anodic electrodeposition, thermal oxidation and others. In a number of embodiments, first electrode 14 includes platinum and iridium oxide. For such embodiments, the platinum can be deposited on the substrate 12, and the iridium oxide can be formed or deposited on the platinum. According to other embodiments, the first electrode 14 includes chromium and iridium oxide. For such embodiments, the chromium can be formed on the substrate 12, and the iridium oxide can be formed on the chromium. According to other embodiments, the first electrode 14 includes titanium and iridium oxide. For such embodiments, the titanium can be formed on the substrate 12, and the iridium oxide can be formed on the titanium. The first electrode 14 is positioned so that it comes into contact with the sample solution/electrolyte (for example, within a sample tissue) of which the pH is to be measured.

Second electrode 16 functions as a reference electrode, and may include any suitable type of material. Desirably, reference electrode 16 maintains a constant or substantially constant potential in the electrolyte solution. In a number of embodiments, second electrode 16 includes platinum and silver. For such embodiments, the platinum can, for example, be formed or deposited on substrate 12, and the silver can be formed or deposited on the platinum. According to other embodiments, second electrode 16 includes platinum and silver chloride. For such embodiments, the platinum can, for example, be formed or deposited on substrate 12, and the silver chloride can be formed or deposited on the platinum. According to other embodiments, second electrode 16 includes chromium and silver. For such embodiments, the chromium can, for example, be formed or deposited on the substrate 12, and the silver can formed on the chromium. According to other embodiments, second electrode 16 includes chromium and silver chloride. For such embodiments, the chromium can, for example, be formed or deposited on substrate 12, and the silver chloride can be formed on the chromium. According to other embodiments, second electrode 16 includes titanium and silver. For such embodiments, the titanium can, for example, be formed or deposited on substrate 12, and the silver can be formed on the titanium. According to other embodiments, second electrode 16 includes titanium and silver chloride. For such embodiments, the titanium can, for example, be formed or deposited on the substrate 12, and the silver chloride can be formed or deposited on the titanium. Second electrode 16 is positioned so that it is in contact with a reference solution within fluidic closed loop channel 20.

Bubble transport system 18 and bubbles 30 and 32 operate in connection with liquid junction 22 and the reference analyte solution within fluidic channel 20 as a fluidic switch or controller 19. Fluidic switch 19 is, for example, operable to place pH sensor 10 in an on state or in an off state. Fluidic switch 19 may be any type of fluidic switch suitable to provide a barrier between a fluid transporting member such a liquid junction 22 and the reference electrolyte solution. In a number of embodiments, fluid switch 19 is operable to turn pH sensor (or another device) off and on by, for example, disrupting the ionic electrical connection between the analyte solution and the reference solution. Fluid switch 19 can also be operable to reduce or eliminate mass transfer between the analyte solution and the reference solution.

In a number of embodiments, as described in more detail hereinafter, bubble transport system 18 can, for example, use electrowetting-on-dielectric principles to effect switching functionality. According to various embodiments, bubble transport system 18 can, for example, include a plurality of electrodes. In the illustrated embodiment, bubble transport system includes three electrodes 18a, 18b and 18c. Bubble transport system 18 may include any suitable type of material. In various embodiments, bubble transport system 18 includes platinum, an insulating layer (e.g., silicon oxide, parylene, etc.), and a hydrophobic layer (e.g., a fluorocarbon hydrophobic layer). In such embodiments, the platinum can, for example, be formed or deposited on substrate 12, and the insulating layer and the hydrophobic layer can be formed or deposited on the platinum. According to other embodiments, bubble transport system 18 includes chromium, an insulating layer, and a hydrophobic layer. For such embodiments, the chromium can, for example, be formed or deposited on substrate 12, and the insulating layer and the hydrophobic layer can be formed or deposited on the chromium. Bubble transport system 18 is positioned so that it is in direct contact with the reference solution of fluidic closed loop channel 20.

In the representative embodiment of FIGS. 1 and 2, fluidic channel 20 is a closed loop channel 20 which is collectively defined by substrate 12 and cover 24. Fluid closed loop channel 20 can, for example, include any suitable type of ionically conductive aqueous solution. For example, according to various embodiments, fluidic channel 20 includes a saturated potassium chloride solution. According to other embodiments fluidic channel 20 includes a saturated silver chloride solution. Fluidic channels hereof need not be closed loop fluid channels. The fluidic channels enable movement of or one or more bubbles or, in the case where a bubble is generated within the channel as described below, the fluidic channel allows displacement of the liquid so that the one or more bubbles can be formed to a desired volume.

As shown in FIG. 1, in a number of embodiments, fluidic closed loop channel 20 surrounds bubble transport system 18, and includes a first bubble 30 and a second bubble 32. First bubble 30 is hydrodynamically connected to the second bubble 32 via the saturated reference solution. Thus, when first bubble 30 is driven from a first position to a second position, second bubble 32 moves from a third position to a fourth position. The third position is shown in solid lines in FIGS. 1 and 2, while the fourth position is show in dashed lines in FIGS. 1 and 2. Accordingly, first bubble 30 may be considered a “master” bubble and second bubble 32 may be considered a “slave” bubble. First and second bubbles 30 and 32 may, for example, include any suitable type of fluid material immiscible in the reference solution. At least bubble 32 can, for example, be immiscible in the analyte solution. For example, according to various embodiments, first and second bubbles 30 and 32 may include air, oil, a gas other than air (for example, hydrogen, oxygen, a mixture of oxygen and hydrogen, etc), etc.

As used herein, the term “bubble” refers to a globule or volume of one substance (a fluid) in another fluid (the reference electrolyte solution). A bubble can, for example, be formed of a gas that is immiscible in the liquid within channel 20 (that is, the saturated reference solution) or a liquid that is immiscible in the liquid within channel 20.

Liquid junction 22 is positioned between the sample or analyte electrolyte solution and the reference solution enclosed in fluidic closed loop channel 20 (for example, saturated potassium chloride), and provides for ionic electrical connection between the analyte electrolyte solution and the reference solution in fluidic closed loop channel 20. In a number of embodiments, liquid junction 22 is a member through which fluid transport can occur and may, for example, include a porous or permeable material. For example, according to various embodiments, liquid junction 22 includes a hydrophilic porous polymer. A porous material for liquid junction 22 can, for example, have a pore size of less than one micrometer. In a number of embodiments, liquid junction 22 is designed to limit or minimize mass exchange between the solution in the fluidic closed loop channel 20 and the sample electrolyte solution (for example, by limiting pore size in the case of a porous material). As shown in FIG. 2, liquid junction 22 is positioned between the substrate 12 and the cover 24 in the illustrated embodiment.

Cover 24 is connected to substrate 12, and cooperates with substrate 12 to define fluidic closed loop channel 20. Cover 24 may, for example, include any suitable type of impermeable material. In the case of an implantable pH sensor 10 cover 24 (and other components of pH sensor 10 which contact an organism) can, for example, be biocompatible. For example, according to various embodiments, cover 24 includes glass or polydimethylsiloxane. Cover 24 may be connected to the substrate 12 in any suitable manner. For example, according to various embodiments, the cover 24 is bonded to the substrate 12. In several embodiment in which cover 24 was glass and substrate 12 was PDMS, cover 24 was readily bonded to substrate 12 by simply pressing them together after O₂ plasma treatment of surfaces. In, for example, cases in which the fluidic channel width is relatively large (for example, about 1 mm or larger) an adhesive can be used to bond cover 24 to substrate 12.

As described above, in the illustrated representative embodiment of FIGS. 1 and 2, a plurality of connection elements or pads 26a through 26e are connected to substrate 12, and may include any suitable type of conductor. For example, according to various embodiments, connection pads 26a-e include platinum. According to other embodiments, connection pads 26a-e include chromium. According to other embodiments, connection pads 26a-e include titanium. According to other embodiments, connection pads 26a-e include gold. Connection pad 26a is connected to the first electrode 14 via conductor 28a. Connection pad 26b is connected to second electrode 16 via conductor 28b. Connection pads 26c, 26d and 26e are connected to electrodes 18a, 18b and 18c of bubble transport system 18 via the conductors 28c, 28d and 28e, respectively. Connection pads 26a-e provide for electrical connection of first electrode 14, second electrode 16, and electrodes 18a, 18b and 18c of fluidic switch 18 to one or more circuits external to the pH sensor 10. As illustrated in FIG. 1, first electrode 14 and second electrode 16 can, for example, be connected to measurement electronics or circuitry 40 which can, for example, include potentiometer circuitry as known in the art. Electrodes 18a, 18b and 18c of bubble transport system 18 can, for example, be in electrical connection with control electronics or circuitry 50.

The plurality of conductors 28a-e may, for example, be formed on a surface of substrate 12, and function to connect first electrode 14, second electrode 16, and electrodes 18a-c to respective connection pads 26a-e. As shown in FIG. 1 and as described above, a first conductor 28a connects first electrode 14a to first connection pad 26a, and a second conductor 28b connects second electrode 16 to second connection pad 26b. Similarly, individual conductors 28c-e connect electrodes 18a-c of bubble transport system 18 to corresponding connection pads 26c-e. respectively. Conductors 28a-e may, for example, include any suitable type of conductive material. For example, according to various embodiments, conductors 28a-e include platinum. According to other embodiments, conductors 28a-e include chromium. According to other embodiments, conductors 28a-e include titanium. According to other embodiments, conductors 28a-e include gold.

In operation of the representative embodiment illustrated in FIGS. 1 and 2, first electrode 14 is exposed to the sample electrolyte (or to a sample tissue). When pH sensor 10 is in an off state (via fluidic switch 19), first bubble 30 is positioned on the “leftmost” (in the orientation the figures) electrode 18a of bubble transport system 18, and second bubble 32 is positioned against liquid junction 22. The positioning of the first bubble 30 and second bubbles 32 may, for example, be realized in any suitable manner. For example, according to various embodiments, electrowetting-on-dielectric techniques may be utilized to move the first bubble 30 and second bubble 32 to the respective positions. For such embodiments, the sequential activation of “rightmost” electrode 18c and “middle” electrode 18b of bubble transport system 18 may be utilized to cause first bubble 30 and second bubble 32 to move to the respective positions associated with the off state of pH sensor 10.

In the off state position, second bubble 32 can, for example, form a barrier over second electrode 16 and liquid junction 22, effectively blocking the fluid/electrical (ionic) connection between the sample electrolyte and the saturated solution in the fluidic closed loop channel 20, thereby reducing or preventing the dissolution of second electrode 16 into the saturated solution, and reducing or preventing mass exchange through liquid junction 22. When second bubble 32 is in the above-described, off-state position, immiscible phase interfaces (for example, gas-liquid or liquid-liquid immiscible interfaces) are formed between second bubble 32 and the sample electrolyte in or at the surface of the pores of liquid junction 22. The interfacial tension between the phases, for example, between a gas and the liquid phase) operates to reduce or block leakage of the sample electrolyte into fluidic closed loop channel 20. Maintaining pH sensor 10 in an off state extends the useful life of pH sensor 10 as compared to a sensor continuously maintained in an on state.

When a pH level is to be measured, pH sensor 10 is switched to an on state. To be switched to the on state, second bubble 32 is moved so that it does not form a barrier over second electrode 16 and the liquid junction 22, and thereby allows for the establishment of an electrical connection between the sample electrolyte and the saturated solution in fluidic closed loop channel 20. According to various embodiments, second bubble 32, which is hydrodynamically connected to first bubble 30, is moved away from second electrode 16 and liquid junction 22 by moving first bubble 30 away from “leftmost” electrode 18a of bubble transport system 18.

First bubble 30 may be moved away from “leftmost” electrode 18a of bubble transport system 18 in any suitable manner. For example, according to various embodiments, electrowetting-on-dielectric principles are utilized to move first bubble 30, which in turn causes movement of second bubble 32. In electrowetting-on-dielectric devices or systems, bubbles are transported by programming and sequentially activating arrays of electrodes.

For such embodiments, the activation of “leftmost” electrode 18a of bubble transport system 18 operates to move first bubble 30 away from “leftmost” electrode 18a of bubble transport system 18 and towards “rightmost” electrode 18c of bubble transport system 18. The movement of first bubble 30 towards the “rightmost” electrode 18c of bubble transport system 18 causes second bubble 32 to move away from second electrode 16 and liquid junction 22, thereby removing the barrier over second electrode 16 and liquid junction 22. The removal of the barrier allows for the establishment of the fluid/electrical (ionic) connection between the sample electrolyte and the saturated solution in fluidic closed loop channel 20.

In the manner described above, pH sensor 10 can be quickly switched between the off and on states, with very low energy consumption. By forming a barrier over second electrode 16 and liquid junction 22 during the off state, and exposing second electrode 16 and liquid barrier 22 to the saturated reference solution of the fluidic closed loop channel 20 only during the on state, dissolution of the second electrode 16 and mass exchange through the liquid junction 22 is reduced or minimized, thereby increasing the useful life of pH sensor 10.

As illustrated schematically in FIG. 1, at least one power source 60 such as a battery can be provided in electrical connection with sensor electronics 40 and control electronics 50. Power source 60 can, for example be used to power sensor electronics 40, control electronics 50 and bubble transport system 18 in the embodiment of FIG. 1. In a number of embodiments, pH sensor 10 can, for example, be actuatable and/or controllable via an external device 70 which communicates (for example, wirelessly via, for example, a radio frequency or RF signal) with, for example, a transceiver 52 in communicative connection with control electronics 50. Control electronics 50 can, for example, be programmed (for example, via one or more programmed processors) to cause bubbles 30 and 32 to move as describe above to enable pH sensor 10 to measure pH at some predetermined time cycle and/or in response to an external signal (for example, external to a body in which pH sensor 10 is implanted). When pH sensor 10 is activated or enabled, a pH reading is acquired by sensor electronics 40. Sensor electronics 40 is in communicative connection with control electronics 50 which effects control of bubble transport system 18. Once a measurement is obtained, pH sensor 10 can be placed in the off state or inactivated via control of bubble transport system 18 as described above. The measured pH value can, for example, be made available for use (for example, either for transmission to outside the body via transceiver 52, or for use by another implanted system, which can, for example, include a treatment device).

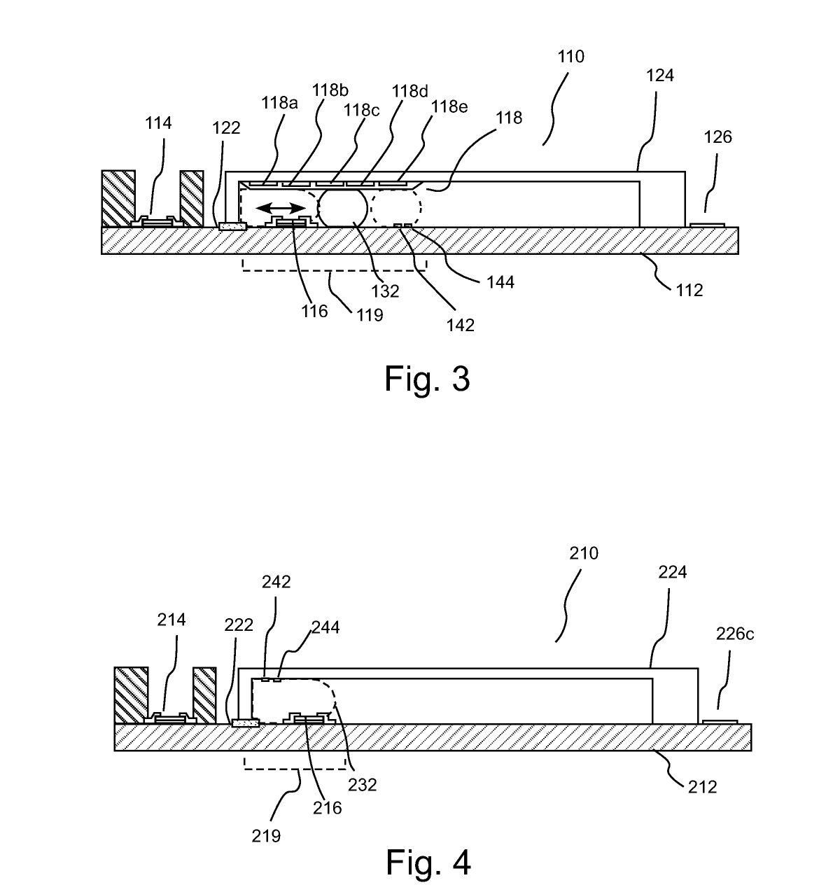

In several embodiments of the present invention, a pH sensor includes a single bubble to effect switching between an on state and an off state. For example FIG. 3 illustrates another representative embodiment of a pH sensor 110 in which a single bubble 132 within a channel 120 (formed between a cover 124 and a substrate 112) is used to form a barrier over a second or reference electrode 116 and a liquid junction 122 (as described in connection with pH sensor 10) to operate as a fluidic switch or controller 119. As described above, by forming or creating a barrier covering liquid junction 22, an off-state is created, wherein ionic electrical connection between the analyte solution (which contacts first or indicating electrode 114) and the reference solution within channel 20 is disrupted or prevented. Furthermore, in the off-state, bubble 132 reduces, minimizes or eliminates mass transfer between the analyte solution and the reference solution. The off state further reduces dissolution of electrode 116 within the reference solution. If bubble 32 is of sufficient size to cover electrode 116, dissolution (mass transfer) between electrode 116 and the reference solution can be further reduced, minimized or eliminated. Dissolution of second electrode 116 and mass exchange through the liquid junction 122 thus occurs to a significant extent only during the on state, thereby increasing the useful life of pH sensor 110.

In the embodiment of FIG. 3, gas bubble 32, which is a mixture of oxygen and hydrogen, is generated via electrolysis using an anode 142 and a cathode 144 that are positioned relatively close to each other (for example, within approximately 4 μm in several embodiments). In the embodiment illustrated in FIG. 3, bubble transport system 118 (for example, an electrowetting-on-dielectric system) is positioned on a top surface of fluidic channel 120. Bubble transport system 118, can, for example, include an array of electrodes 118a-e, which are positioned on an inner surface of cover 124 (that is, on a top surface of fluidic channel 120). In the illustrated embodiment, the electrolysis electrodes used to create bubble 132 (that is, anode 142 and cathode 144) are placed on substrate 112. To create bubble 132 (or a plurality of bubbles as, for example, discussed in connection with pH sensor 10), one can, for example, apply a potential difference of approximately 5 V between and anode/cathode pair such as anode 142 and cathode 144.

In operation of fluidic switch 119, bubble 132 is first generated via electrolysis using anode 142 and cathode 144 (see rightmost dashed lines in fluidic channel 120). The size of the bubble created can, for example, be controlled via control of the time that a potential is applied. To place fluid switch 119 in an off state, bubble 132 is transported via bubble transportation system 118 to cover liquid junction 122 (see leftmost dashed lines in fluidic channel 120) and, in several embodiments, to cover reference electrode 122. To place fluid switch in an on state, bubble 132 is transported via bubble transportation system 118 so that is does not cover either liquid junction 122 or reference electrode 116.

FIG. 4 illustrates another representative embodiment of a pH sensor 210 in which a single bubble 232 within a channel 220 (formed between a cover 224 and a substrate 212) is used to form a barrier over a second or reference electrode 216 and a liquid junction 222 as described in connection with pH sensors 10 and 110 to operate as a fluidic switch 219. As described above, by forming or creating a barrier covering liquid junction 222, an off-state is created, wherein ionic electrical connection between the analyte solution (which contacts first or indicating electrode 214) and the reference solution within channel 220 is disrupted or prevented. If bubble 232 is of sufficient size to cover electrode 216, dissolution (mass transfer) between electrode 216 and the reference solution can be further reduced, minimized or eliminated.

In operation of fluidic switch 219, bubble 232 is first generated via electrolysis using anode 242 and cathode 244 (see rightmost dashed lines in fluidic channel 120). As described above, the size of the bubble created can, for example, be controlled via control of the time that a potential is applied. To place fluid switch 219 in an off state, bubble 232 is generated to a size to cover liquid junction 222 and, in several embodiments, to cover reference electrode 222. To subsequently place fluid switch in an on state, bubble 232 is reduced in size or completely eliminated via reversing of the electrolysis process using anode 242 and cathode 244 so that it does not cover either liquid junction 122 or reference electrode 116. To effect bubble reduction or elimination, catalysis can be used to lower the energy barrier in the reverse process. For the case of bubble 232 including hydrogen and oxygen bubble. platinum (Pt) can, for example, be used as a catalyst. In a number of embodiments, anode 242 and cathode 244 can, for example, be made to include a catalytic material such as Pt. When an electric potential is applied to the anode 242 and cathode 244, bubble 32 grows. When the electric potential is shut off, bubble 232 shrinks In an alternative embodiment, a source of a catalyst such as Pt can be provided separately from anode 242 and cathode 244.

Fluidic switches or controller such as fluidic switches or controllers 19, 119 and 219 can, for example, be used in other devices wherein it is desirable to control fluid connection, ionic conduction and/or mass transfer across a member though which a fluid can be transported (for example, a porous or permeable member such as a porous polymeric member, a permeable membrane etc).

The foregoing description and accompanying drawings set forth a number of examples of representative embodiments at the present time. Various modifications, additions and alternative designs will become apparent to those skilled in the art in light of the foregoing teachings without departing from the spirit hereof, or exceeding the scope hereof, which is indicated by the following claims rather than by the foregoing description. All changes and variations that fall within the meaning and range of equivalency of the claims are to be embraced within their scope.

What is claimed is:

1. A pH sensor, comprising:
an enclosed fluidic channel;
an electrolyte solution within the fluidic channel;
a first electrode exterior to the fluidic channel;
a second electrode within the fluidic channel;
a liquid junction extending between the fluidic channel and an exterior of the fluidic channel, the liquid junction adapted to provide fluid connection between the electrolyte solution within the fluidic channel and an exterior of the fluidic channel; and
a fluidic switch in operative connection with the liquid junction to control whether the liquid junction provides fluid connection between the electrolyte solution within the fluidic channel and the exterior of the fluidic channel.

2. The pH sensor of claim 1 further comprising:
a substrate; and
a cover connected to the substrate, wherein the cover and the substrate cooperate to define the fluidic channel.

3. The pH sensor of claim 1 wherein the fluidic switch comprises at least a first bubble within the fluidic channel.

4. The pH sensor of claim 3 further comprising a bubble transportation system to transport the first bubble between a first position wherein it contacts the liquid junction and a second position wherein it does not contact the liquid junction.

5. The pH sensor of claim 4 wherein the bubble transportation system comprises electrowetting-on-dielectric system comprising an array of electrodes.

6. The pH sensor of claim 4 wherein the first bubble contacts the liquid junction and the second electrode in the first position and does not contact the liquid junction or the second electrode in the second position.

7. The pH sensor of claim 3 further comprising a bubble transportation system to transport the first bubble between a first position wherein the first bubble forms a barrier between the liquid junction and electrolyte solution and forms a barrier between the second electrode and the electrolyte solution and a second position wherein the bubble does not form a barrier between the liquid junction and electrolyte solution and does not form a barrier between the second electrode and the electrolyte solution in the second position.

8. The pH sensor of claim 4 further comprising an electrode system in fluid connection with the reference electrolyte solution within the fluidic channel to generate the first bubble within the fluidic channel.

9. The pH sensor of claim 1 further comprising an electrode system in fluid connection with the reference electrolyte solution within the fluidic channel to generate at least one bubble within the fluidic channel so that the bubble can contact the liquid junction and a system to reduce the size of the bubble so that the bubble does not contact the liquid junction.

10. The pH sensor of claim 9 wherein the system to reduce the size of the bubble comprises a catalyst on the electrode system.

11. The pH sensor of claim 10 wherein the bubble is generated to contact the liquid junction and the second electrode in the first position and then reduced in size so that the bubble does not contact the liquid junction or the second electrode.

12. The pH sensor of claim 1 further comprising an electrode system in fluid connection with the reference electrolyte solution within the fluidic channel to generate at least one bubble within the fluidic channel so that the bubble can form a barrier between the liquid junction and electrolyte solution and form a barrier between the second electrode and the electrolyte solution and a system to reduce the size of the bubble so that the bubble does not form a barrier between the liquid junction and electrolyte solution and does not form a barrier between the second electrode and the electrolyte solution.

13. The pH sensor of claim 2, wherein the substrate comprises a glass or a polymer and the cover comprises a glass or a polymer.

14. The pH sensor of claim 1, wherein the first electrode comprises at least one of platinum, chromium, titanium, or iridium oxide.

15. The pH sensor of claim 1, wherein the second electrode comprises at least one of platinum, chromium, titanium, silver, and silver chloride.

16. The pH sensor of claim 2, wherein the cover comprises polydimethylsiloxane.

17. The pH sensor of claim 4, wherein the fluidic switch comprises at least a second bubble spaced from the first bubble and hydrodynamically connected to the first bubble via the reference solution.

18. The pH sensor of claim 1, wherein the electrolyte solution comprises at least one of a potassium chloride solution or a silver chloride solution.

19. The pH sensor of claim 3, wherein the first bubble comprises a fluid immiscible in the electrolyte solution.

20. The pH sensor of claim 1, wherein the liquid junction comprises a porous polymer.

21. The pH sensor of claim 1 wherein the pH sensor is adapted to be implantable within a body.

22. The pH sensor of claim 1 wherein the pH sensor is a microscale pH sensor.

23. The pH sensor of claim 1 wherein the pH sensor has dimensions less than one centimeter.

24. A fluidic controller, comprising:
an enclosed fluidic channel
a liquid within the fluidic channel;
a liquid junction extending between an interior of the fluidic channel and an exterior of the fluidic channel; and
at least a first bubble within the fluidic channel.

25. The fluidic controller claim 24 further comprising a bubble transportation system to transport the first bubble between a first position wherein it contacts the liquid junction and a second position wherein it does not contact the liquid junction.

26. The fluidic controller of claim 25 wherein the bubble transportation system comprises an electrowetting-on-dielectric system comprising an array of electrodes.

27. The fluidic controller of claim 25 further comprising an electrode system in fluid connection with the reference electrolyte solution within the fluidic channel to generate the first bubble within the fluidic channel.

28. The fluidic controller of claim 24 further comprising an electrode system in fluid connection with the liquid within the fluidic channel to generate the first bubble within the fluidic channel so that the first bubble can contact the liquid junction and a system to reduce the size of the bubble so that the first bubble does not contact the liquid junction.

29. The fluidic controller of claim 28 wherein the system to reduce the size of the bubble comprises a catalyst on the electrode system.

30. A method of controlling fluid connection between a liquid in an enclosed fluidic channel and an exterior of the fluidic channel, wherein the fluidic channel comprises a liquid junction extending between the fluidic channel and the exterior of the fluidic channel and the liquid junction is adapted to provide fluid connection between fluidic channel and the exterior of the fluidic channel, comprising: controllably positioning a bubble of a fluid immiscible in the liquid to be in contact with the liquid junction.
